(19) United States
(12) Patent Application Publication
(10) Pub. No.: US 2025/0204568 A1
(43) Pub. Date: Jun. 26, 2025

(54) CANNABINOID-CONTAINING COMPOSITIONS AND USES THEREOF IN FOOD

(71) Applicant: BUZZELET DEVELOPMENT AND TECHNOLOGIES LTD., Or Akiva (IL)

(72) Inventors: Aharon EYAL, Jerusalem (IL); Dana BERNEMAN ZEITOUNI, Kfar Yona (IL)

(21) Appl. No.: 18/845,450

(22) PCT Filed: Mar. 12, 2023
(86) PCT No.: PCT/IB2023/052363

§ 371 Date: Sep. 10, 2024

Related U.S. Application Data
(60) Provisional application No. 63/319,400, filed on Mar. 14, 2022.

(51) Int. Cl. A23L 33/105; A23L 33/125; A23L 33/21

(52) U.S. Cl. CPC A23L 33/105; A23L 33/125; A23L 33/21

(57) ABSTRACT

Provided is a composition comprising (a) at least 50% wt carbohydrate, (b) less than 35% wt water (c) water activity in the range between 0.65 and 0.9, (d) at least one cannabinoid at a concentration of at least 0.1% wt of said composition; (e) a thickener, wherein the composition comprises less than 2% wt fat. Further provided are food products comprising the composition.

### CROSS-REFERENCE TO RELATED APPLICATIONS

The present application gains priority from U.S. Provisional Patent Application Ser. No. 63/319,400 filed Mar. 14, 2022 which is incorporated by reference as if fully set-forth herein.

### FIELD OF THE INVENTION

The present invention relates to the field of cannabinoid-containing compositions, and more specifically to compositions comprising a cannabinoid, a carbohydrate and a thickener, and uses of such compositions as food ingredients in food products.

### BACKGROUND OF THE INVENTION

It is known in the art to administer cannabinoids to a subject in the form of a food product. However, in order to provide a sufficiently high daily dosage of cannabinoid to produce a desired therapeutic effect, it may be necessary to consume an unreasonably large amount of the food product. Alternatively, cannabinoids may be provided in the food product at high concentrations. However, high concentrations of cannabinoid are generally associated with an undesirable flavor, which may be particularly problematic if the subject is a child, or a subject having low compliance.

There is thus an unmet need for a cannabinoid-containing food product which provides cannabinoid at a desired concentration and dosage level and is devoid of an undesirable flavor.

### SUMMARY OF THE INVENTION

According to an aspect of some embodiments of the present invention, there is provided a composition comprising (a) at least 50% wt carbohydrate, (b) less than 35% wt water (c) water activity in the range between 0.65 and 0.9, (d) at least one cannabinoid at a concentration of at least 0.1% wt of said composition; (e) a thickener, wherein the composition comprises less than 2% wt fat.

According to a further aspect of some embodiments of the present invention, there is provided a food product comprising the composition as disclosed herein.

According to a further aspect of some embodiments of the present invention, there is provided a food product comprising the food ingredient as disclosed herein.

### DETAILED DESCRIPTION OF THE INVENTION

The present invention relates to compositions comprising a cannabinoid, a carbohydrate and a thickener, and uses of such compositions as food ingredients in food products.

Cannabinoids are lipophilic and can therefore be provided at relatively high concentrations in compositions comprising fats. However, it has been found that fats enhance the undesirable flavor associated with cannabinoids, particularly at high concentrations.

The present inventors have surprisingly found that cannabinoids may be provided at high concentrations in compositions having low or no fat content, which are devoid of an undesirable flavor, in compositions comprising a high carbohydrate content, low water activity and a thickener.

Unless otherwise defined, all technical and scientific terms used herein have the same meaning as commonly understood by one of ordinary skill in the art to which this invention belongs.

The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

As used herein, the term “treating” includes ameliorating, mitigating, and reducing the instances of a disease or condition, or the symptoms of a disease or condition.

As used herein, the term “therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

Unless otherwise indicated, all numbers expressing quantities, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”. As used herein, the terms “water content” and “moisture content” are used interchangeably.

Unless indicated otherwise, percent is weight percent and ratio is weight/weight ratio.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a composition comprising (a) at least 50% wt carbohydrate, (b) less than 35% wt water (c) water activity in the range between 0.65 and 0.9, (d) at least one cannabinoid at a concentration of at least 0.1% wt of said composition; (e) a thickener, wherein the composition comprises less than 2% wt fat.

According to some embodiments, the composition comprises 50% wt, about 55% wt, about 60% wt, about 65% wt, about 70% wt, about 75% wt, or even greater than 75% wt of carbohydrate.

According to some embodiments, the composition comprises about 32% wt, about 29% wt, about 26% wt, about 23% wt, about 20% wt, about 17% wt, or even less than 17% wt of water.

According to some embodiments, the composition comprises 0.1% wt, about 0.5% wt, about 1% wt, about 1.5% wt, about 2% wt, about 2.5% wt or up to about 3% wt of each one of one or more cannabinoids.

According to some embodiments, the composition comprises one cannabinoid. According to some embodiments, the composition comprises two, three, four or more than four cannabinoids. According to some embodiments, the composition comprises 0.1% wt, about 0.5% wt, about 1% wt, about 1.5% wt, about 2% wt, about 2.5% wt, about 3w % wt, about 3.5% wt, about 4% wt, about 4.5% wt or up to about 5% wt total weight of cannabinoids. According to some embodiments, the cannabinoid comprises or consists of cannabidiol.

According to some embodiments, the composition comprises less than 2% wt, less than 1.5% wt, less than 1% wt, less than 0.5% wt, less than 0.2% wt or about 0% wt fat. In some such embodiments, the composition is devoid or substantially devoid of fat.

According to some embodiments, the composition is homogenous. As used herein, the term “homogenous” refers to a composition comprising a mixture of components wherein the components are uniformly distributed throughout the mixture.

According to some embodiments, the composition is devoid or substantially devoid of an emulsion.

As used herein, the term “substantially devoid” with regard to a component is intended to mean lacking any detectable amount of such a component.

According to some embodiments, the carbohydrate comprises at least one selected from the group consisting of sucrose, glucose, fructose and combinations thereof.

According to some embodiments, the thickener comprises pectin.

According to some embodiments, the composition further comprises a flavorant (also referred to as a flavoring agent). As used herein, the term “flavorant” or “flavoring agent” refers to an additive to a food product which alters the natural flavor or a food product or provides a flavor for a food product which lacks a desired flavor.

According to some embodiments, the composition further comprises a high intensity sweetener. As used herein, the term “high intensity sweetener” refers to a sugar substitute or a sugar derivative having many times the sweetness of sucrose. According to some such embodiments, the high intensity sweetener is selected from the group consisting of an FDA-approved sugar substitute, such as aspartame, sucralose, neotame, acesulfame potassium, saccharin and advantame, stevioside or combinations thereof.

According to some embodiments, the composition further comprises a dietary fiber. According to some such embodiments, the dietary fiber is derived from a source selected from the group consisting of a whole-grain, a fruit, a vegetable, a legume and a nut or seed.

According to some embodiments, the composition further comprises a food-grade antioxidant. According to some such embodiments, the antioxidant is selected from the group consisting of vitamin A, vitamin C, vitamin E, coenzyme Q10, manganese, iodide, selenium, a carotenoid (such as alpha-carotene, astaxanthin, beta-carotene, canthaxanthin, cryptoxanthin, lutein, lycopene, lutein and zeaxanthin, or combinations thereof), a flavonoid (such as a flavone, a flavonol, a flavanone, a flavonol, an isoflavone phytoestrogen, a stilbenoid, an anthocyanin, or combinations thereof), a phenolic acid or ester thereof, curcumin, a flavonolignan, xanthone, eugenol, a polyphenol, capsaicin, bilirubin, citric acid, oxalic acid, phytic acid, N-acetylcysteine, lipoic acid, or combinations thereof.

According to some embodiments, the composition further comprises at least one terpene, such as one, two, three, four, five or more than five terpenes, at a total terpenes to total cannabinoids weight/weight ratio of 0.05 to 0.5. According to some such embodiments, the terpene is a cannabis terpene. According to some embodiments, the terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to some embodiments, the composition is spreadable. As used herein, the term “spreadable” refers to a composition which may be spread as a layer, such as a coating on a layer of an edible solid material, including but not limited to a biscuit, cracker, cake, a slice of bread or the like.

According to some embodiments, the spreadable composition may be spread as a filling between two layers of an edible solid material, such as two crackers, biscuits, slices of bread or the like.

According an aspect of some embodiments of the present invention, there is provided a food product comprising the spreadable composition as disclosed herein.

According to some embodiments, the food product further comprises at least one layer of an edible solid material, wherein the spreadable composition is provided as a coating layer on the at least one layer of edible solid material.

According to some embodiments, the food product further comprises at least a first and a second solid layer of an edible solid material, wherein the spreadable composition is provided between the first and the second layer of edible solid material.

According to some embodiments, the food product comprises the spreadable composition as a filling, such as a filling of a cupcake, muffin, pie, tart, candy or candy bar, or the like.

### Examples

Cookies comprising cannabidiol (CBD) were prepared, wherein each cookie had a weight of about 600 mg.

### Example 1: Vanilla Cream Cookies

Ingredients:

Plain gluten-free sandwich cookies

Commercial vanilla cream filling comprising 30wt % water, sugar and starch, optionally with additional ingredients such as food colorants, preservatives, food acids, flavorants, as desired.

Method of preparation:

0.4 g isolated CBD and 19.6 g vanilla cream were mixed and heated slightly in a microwave, then mixed again to achieve a homogenous mixture. 6.5 g of this mixture was spread on a plain gluten-free sandwich cookie as a filling layer. An additional plain gluten-free sandwich cookie was placed on top of the filling layer.

### Example 2: Strawberry Cream Cookies

Ingredients:

Plain gluten-free cookies.

Commercial strawberry jam comprising 30wt % water, sugar and starch, optionally with additional ingredients such as food colorants, preservatives, food acids, flavorants, as desired.

Method of preparation:

0.4 g isolated CBD and 19.6 strawberry jam were mixed and heated slightly in a microwave, then mixed again to achieve a homogenous mixture. 6.5 g of this mixture was spread on a plain gluten-free sandwich cookie as a filling layer. An additional plain gluten-free sandwich cookie was placed on top of the filling layer.

### Example 3: Chocolate Chip Cookies

Ingredients:

Plain gluten-free cookies.

Chocolate chips or gluten-free chocolate cookies, comprising gluten-free flour and sugar.

Method of preparation:

30% isolated CBD MCT oil was prepared and heated at 70° C. for about 10 minutes.

0.2 g of CBD was applied such as by dripping slowly, to each cookie. The cookies were allowed to stand for 24 hours in order to absorb the oil and the flavors.

1. A composition comprising (a) at least 50% wt carbohydrate, (b) less than 35% wt water (c) water activity in the range between 0.65 and 0.9, (d) at least one cannabinoid at a concentration of at least 0.1% wt of said composition; (e) a thickener, wherein the composition comprises less than 2% wt fat.

2. The composition of claim 1, wherein said the composition is homogenous.

3. The composition of claim 1, devoid of an emulsion.

4. The composition of claim 1, wherein said carbohydrate comprises at least one selected from the group consisting of sucrose, glucose, fructose and combinations thereof.

5. The composition of claim 1, wherein said cannabinoid comprises cannabidiol.

6. The composition of claim 1, wherein said thickener comprises pectin.

7. The composition of claim 1, further comprising a flavorant.

8. The composition of claim 1, further comprising a high intensity sweetener.

9. The composition of claim 1, further comprising a dietary fiber.

10. The composition of claim 1, further comprising a food-approved antioxidant.

11. The composition of claim 1, further comprising at least one terpene at a total terpenes to total cannabinoids weight/weight ratio of 0.05 to 0.5.

12. The composition of claim 11, wherein said terpene is a cannabis terpene.

13. The composition of claim 1, wherein the composition is spreadable.

14. A food product comprising the spreadable composition of claim 13.

15. The food product of claim 14, further comprising at least one layer of an edible solid material, wherein the spreadable composition is provided as a coating layer on said at least one layer of said edible solid material.

16. The food product of claim 14, further comprising at least a first and a second solid layer of an edible solid material, wherein the spreadable composition is provided between said first and said second layer of said edible solid material.
